(19) United States
(12) Patent Application Publication
(10) Pub. No.: US 2016/0354418 A1
(43) Pub. Date: Dec. 8, 2016

(54) USE OF COMPOSITIONS COMPRISING BIFIDOBACTERIUM ANIMALIS SSP. LACTIS LMG P-28149

(71) Applicant: VESALE PHARMA SA, Noville-sur-Mehaigne (BE)

(72) Inventors: Johan Quintens, Hoegaarden (BE); Jehan Lienart van Lidth de Jeude, Bigonville (LU); Corinne Grangette, Bigonville (LU); Bruno Pot, Sint-Michiels (BE); Kassem Makki, Linselles (FR); Isabelle Wolowczuk, La Madeleine (FR); Jeanne Alard, La Neuville aux Joûtes (FR); Véronique Lehrter-Valenti, Nanteuile la Forêt (FR)

(73) Assignee: VESALE PHARMA SA, Noville-sur-Mehaigne (BE)

(21) Appl. No.: 15/118,077

(22) PCT Filed: Feb. 13, 2015
(86) PCT No.: PCT/EP2015/053166

§ 371 Date: Aug. 10, 2016

(30) Foreign Application Priority Data
Feb. 14, 2014 (BE) 2014/0101
Apr. 25, 2014 (BE) 2014/0292
Apr. 25, 2014 (FR) 1453775

(51) Int. Cl. A61K 35/745; A23L 33/135; C12N 1/20; A61K 8/99; A61K 8/97

(52) U.S. Cl. CPC A61K 35/745; A61K 8/99; A61K 8/975; C12N 1/20; A23L 33/135; A61K 2800/85; A23V 2002/00; A23Y 2300/21

(57) ABSTRACT

A composition made from at least one probiotic for the use of same in the curative treatment of body weight gain in an overweight human and/or in the preventive treatment of body weight gain in a human who is overweight or has been overweight, said probiotic being Bifidobacterium animalis ssp. Iactis n° LMG P-28149.

The present invention relates to a composition based on at least one probiotic for its use in the curative treatment of body weight gain in overweight human beings and/or in the preventive treatment of body weight gain in overweight human beings or having been overweight.

By the terms of [symbol] overweight [symbol] , is meant in the sense of the present invention that the human being has abnormal or excessive accumulation of body fat, mainly abdominal fat, which has a risk for his/her health. Within the scope of the present invention, the term of [symbol] overweight [symbol] also comprises the particular and extreme case of the obesity condition in human beings.

By the terms of [symbol] having been overweight [symbol] , it should be understood in the sense of the present invention that the human being has been subject to a weight loss and has a weight considered as normal (based on the BMI or waist line/hip ratio) from a period comprised between 3 and 5 years.

It is difficult to elaborate a simple index which allows measurement of the overweight and the obesity at the level of a panel of a given population. For example, the scale used for measuring the overweight and obesity index in children and in teenagers, because of the fact that their body undergoes a certain number of physiological changes related to their growth, differs from the one used in adults. Consequently, depending on age, the WHO (World Health Organization) makes available to the general public, different methods for measuring body weight depending on the age of the individual or on the average age of the sample of individuals.

As an illustration, overweight may be evaluated in adults on the base of the Body Mass Index (BMI), which represents a simple measurement of weight as compared relatively to the size currently used for estimating the overweight and the obesity in adults. It corresponds to the weight divided by the square of the size, expressed in kg/m².

The BMI has the advantage of being applied to both genders and to all the adult age intervals. Therefore it should be considered as an approximate indication since it does not necessarily correspond to the same percentage of fat mass depending on the individuals.

In this context, the WHO defines, on the basis of the BMI, the following threshold values for determining whether an adult has overweight or obesity:
a normal weight condition corresponds to a BMI comprised between 20 and 25;
an overweight condition corresponds to a BMI comprised between 25 and 30; and
an obesity condition corresponds to a BMI equal to or greater than 30.

However, it should be noted that the BMI does not give any indication as to the distribution of the abdominal fat relatively to the subcutaneous fat.

This index, even if it has the advantage of making the classification unform to both genders and to all the age intervals of adult individuals, may preferably be interpreted complementarily with another indicator which may for example be the measurement of the waistline of the individual.

Thus an adult male individual having a waistline of greater than 100 cm is considered as being overweight. This threshold is 88 cm in adult women.

This parameter may be generalized to all the age intervals by measuring the waistline (A)/hip circumference (H) ratio.

Overweight is observed for a ratio greater than 1 in male individuals and greater than 0.85 in female gender individuals.

The term of “treatment” is applied within the scope of this invention as soon as the overweight in the human being and in particular human obesity has been recognized as a disease by the WHO.

By the term of [symbol] curative [symbol] , it should be understood in the sense of the present invention, the action which aims at reducing the body weight of an overweight human being and/or at reducing the body weight gain in overweight human beings. The curative treatment therefore implies the action which aims at reaching, preferably during and/or after a diet, a body weight considered as normal in human beings, i.e. with lesser risk for health, in a range of predetermined values, for example based on the BMI, which is comprised between 20 and 25, preferably equal to 22.

By the terms of [symbol] lesser risk for health [symbol] , it should be interpreted within the scope of the present invention that, at a body weight considered as normal, the risk of developing a metabolic syndrome is negligible.

In particular, the curative action mainly aims at reducing abdominal fat in the individuals or at least reduction in the accumulation of abdominal fat.

By the term of [symbol] preventive [symbol] , it should be meant, within the sense of the present invention, the action which aims at stabilizing the body weight of an overweight human being or who has been overweight (i.e. who has lost weight) and therefore at preventing or limiting the gain of body weight in overweight human beings or which have been overweight, preferably during and/or after a diet. The preventive treatment also implies the action which aims at maintaining, preferably during and/or after a diet, a stable body weight, preferably considered as normal in human beings, in a constant range of predetermined values, based for example on the BMI (as a reminder, between 20-25), but the normal body weight may also be interpreted in the light of the A/H ratio of less than 0.85 in individuals of female gender, and less than 1.00 in individuals of male gender.

Presently, on a worldwide scale, overweight and obesity are responsible (because of the associated diseases, like diabetes of type II or cardiovascular diseases), for more deaths than weight insufficiency.

In particular, weight and obesity have attained the proportions of a worldwide epidemic (taken into account that more than 3 million persons at least die each year of pathologies related to their overweight or their obesity) and are no longer limited to so-called rich countries but now also affect countries with low or intermediate revenues. Further it is proven that a high BMI, sometimes jointly with a high A/H ratio, is a factor promoting the metabolic syndrome on the one hand, which is defined within the scope of the present invention as an association of risk factors for health: arterial hypertension, hypertriglyceridemia, low HDL cholesterol level, android obesity condition (for example, an accumulation of abdominal fat), and an increase in glycemia. These indicators in fact form a definition base which is common to the present three main known definitions: the one of the WHO (published in 1998 and then amended in 1999), the one of the National Cholesterol Education Program (NCEP-ATPIII) published in 2001, and that of the International Diabetes Federation published in 2005.

In practice, metabolic syndrome is expressed by the following indicators, in a non-exhaustive way: an abnormally high insulin level; diabetes of type II; hypercholesterolemia (associated with a low level of [symbol] good [symbol] cholesterol HDL); hypertension; a significant increase in the body weight gain over time, especially if this is an abdominal obesity; hypertriglyceridemia; hepatic steatosis; development of a systemic inflammatory condition; and hypertrophy of the adipocytes.

Moreover, the risk of chronic diseases is expressed by the following indicators, in a non-exhaustive way: muscular-skeletal disorders, in particular arthrosis; or certain types of cancers.

The problem of uncontrolled body weight gain leading to overweight in human beings is therefore a proven problem of society having a non-negligible impact on health, on a worldwide level.

It is in this perspective that the WHO has established, in its worldwide strategy for preventing excessive weight gain, recommendations concerning food and physical exercise. Adopted by the World Health Assembly (WHA) in 2004, this strategy defines the steps required for encouraging people to follow a healthy diet and to regularly exercise.

However, if this strategy is based on recommendations preventing weight gain, it does not propose any indications as to the curative and preventive treatments of weight gain in overweight individuals or having been an overweight and which have a risk of regaining weight. Indeed, presently, the problem of uncontrolled weight gain is no longer focussed on individuals of normal weight (i.e. which have a normal BMI and/or a normal waistline) which may gain weight, but especially individuals already overweight from the moment that the WHO indicates that nearly 1.4 billion persons of more than de 20 years old already exhibit overweight. From among the latter, more than 200 million of men and nearly 300 million women are obese, and globally, more than one adult out of ten worldwide is obese.

Therefore there exists a real need for having a treatment which allows reduction or prevention of the overweight or of the obesity in overweight human beings. Preferably, this treatment should not very compelling from the moment that one is aware that a main reason related to overweight and obesity is the lack of diligence to follow a specific diet associated with a treatment which may sometimes be stringent.

In particular, the present invention is a part of a known cause-effect relationship which associates modifications of the intestinal microbiot with the development of obesity (Ley et al., 2006. Nature, 444: 1022-1023; Nadal et al., 2008. Int J. Obes., 33(7): 758-67).

Many studies have been conducted in this context and have supplied the present state of the art including a large panel of documents mentioning the use of treatments based on probiotics within the scope of overweight and obesity.

In particular, it has been shown that the microbiot contributes to maintaining the host in good health. Indeed, the balanced condition of the microbiot contributes to regulation of intestinal homeostasia and to immune equilibrium, so that, any unbalance of the microbiot is interpreted as participating in the development of the metabolic syndrome, in particular in individuals having genetic predisposition for developing this syndrome (Parks et al., Cell Metab, 2012).

The identification of the microbiot—weight gain relationship has been the driving force for a wide panel of studies which have been the subject of various patent applications, the most relevant thereof are commented upon below.

First of all one should mention the international patent application WO2007043933 which proposes the use of strains of Lactobacillus casei F19 and L. acidophilus NCFB 1748, and Bifidobacterium lactis Bb12, in the form of fermented milks, in order to reduce the appetite and the deposition of fats in certain tissues, and thus control the body weight in human beings. However, it seems that it is mostly the joint action of calcium and of dairy proteins which originate in the effects claimed in this application rather than the presence of the aforementioned bacterial strains. Further, this effect only targets the expression of the genes related to the metabolism of the small intestine and therefore does not concern the other organs and tissues involved in the weight gain.

Next, document US20100061967A1 also proposes the use of a composition of bacteria for modulating the expression of the peptides regulating the satiation mechanism, this modulation taking place exclusively in the gastro-intestinal tract.

International patent application WO2009153662 as for it discloses the use of a composition based on Bifidobacteriesa and of Lactobacilli in the treatment of diabetes, a disease coming under the list of indicators associated with the metabolic syndrome induced by overweight or obesity, and this, exclusively based on the capacity which these microorganisms have for reducing inflammation of the peripheral tissues but without acting on the central nervous system, and therefore for example on the central regulation mechanism of satiation.

Further, document US20100150890 gives information on the use of bacterial probiotics in a composition for stimulating the function of the sympathetic nervous system, so that the metabolism, and therefore the energy expense is stimulated. However, it has been shown that the sympathetic tone is also active in certain obese individuals and thus, its activation is not a reliable alternative within the scope of treatment of overweight and obesity.

Patent application US20100111915 exhibits the generic use of a composition of bacterial probiotics as an alternative within the scope of preventing obesity in children. According to US20100111915, this use is based on the bifidogenic effects of probiotics, although this document does not give any tangible base which gives the possibility of establishing that the increase in the number of bifidobacteria in the intestine may be at the origin of this preventive action of the occurrence of obesity in children.

This document US20050112112 proposes the use of a composition of microorganisms generating sugar polymers, which are not digestible by human beings, from monosaccharides and disaccharides present in the gastro-intestinal tract, thereby reducing de facto the absorption of sugars in the body.

Finally, document JP10306028 moreover gives information on an inhibitory action of absorption by the body of cholesterol, and this by the use of bifidobacteria combined with chitosan.

Unfortunately, the use of the compositions of the state of the art, if they allow reduction of overweight and of obesity and reduction of the symptoms associated with the metabolic syndrome (this is referred to for example as remission), it does not allow long-term maintenance for more than 3 to 5 years (this is for example referred to as healing) of the body weight at a desired normal level (i.e. corresponding for example to a BMI between 20 and 25).

Within the scope of the present invention, remission should be distinguished from healing. An individual is said to be in remission if, during medical examinations (weight, waistline measurement, etc.) overweight is no longer detected. But only when healing is referred to after a certain additional time interval, which varies depending on the type of overweight, generally when remission lasts for 3 or 5 years, it is considered that healing has occurred.

The object of the present invention is to overcome the failure of the state of the art by providing a composition as described in the beginning characterized in that said probiotic is Bifidobacterium animalis ssp. lactis no. LMG P-28149.

The strain according to the invention was deposited by the BCCM (Belgian Coordinated Collections of Micro-organisms) and the LMG (Laboratorium voor Microbiologie—Bacte\dot{n}ënverzameling) on Jan. 27, 2014 according to the Treaty of Budapest, under the number of LMG P-28149.

Within the scope of the present invention, the composition preferably comprises the probiotic Bifidobacterium animalis ssp. lactis no. LMG P-28149 in the living condition. By the terms of “in the living condition”, is meant a concentration of living bacteria in the composition comprised between 10⁸ and 10¹³ CFUs per gram of composition.

Within the scope of the present invention, it was observed that this strain of a particular probiotic has a significant positive action on satiation and also a restoration of the intestinal microbiot associated with the limitation of body weight gain.

The role of the presence of the Bifidobacterium animalis ssp. lactis no. LMG P-28149 was notably observed in the recolonization of the intestinal tract by Akkermansia muciniphila, a bacteria notorious for its role in the prevention of obesity in human beings, the level of which is strongly reduced in the overweight or obese individual.

Further, the anti-inflammatory and immuno-regulating action of the strain according to the invention was moreover surprisingly observed, which is associated with the restoration of the expression of the transcription factor PPARγ, involved in the function of T lymphocytes regulating white adipose tissue.

Thus, the composition according to the invention notably allows curative or preventive treatment of the disorder of the intestinal microbiot and of inflammatory diseases associated with this disorder in overweight human beings.

The present composition therefore advantageously aims at curative or preventive treatment of the metabolic syndrome associated with overweight in human beings.

Therefore it was demonstrated, according to the present invention, that the composition based on Bifidobacterium animalis ssp. lactis no. LMG P-28149 notably has the following effects on the body, which are accompanied by a reduction in the body weight gain under a diet rich in fats, preferably during and/or after a diet, or by stabilization of the body weight: an improvement to the insulin sensitivity; a reduction in the development of the abdominal and subcutaneous fat mass; a reduction in the size of the adipocytes; a reduction of the cell infiltration of white adipose tissues, in particular by the pro-inflammatory macrophages; a reduction in the inflammatory markers; a limitation of liver steatosis; and a limitation or reduction of the “bad” cholesterol level.

As this may be seen, the composition according to the invention comprising at least the probiotic Bifidobacterium animalis ssp. lactis no. LMG P-28149 acts on various factors related to overweight.

Within the scope of the present invention, the preventive and curative action on weight gain as well as the action of the metabolic syndrome in overweight human beings was also demonstrated, not only of the specific strain Bifidobacterium animalis ssp. lactis no. LMG P-28149, either in the living condition or not, but also of compounds derived from this strain.

In this context, the composition according to the invention may further comprise at least one or a combination of least two probiotic elements, each element being derived from Bifidobacterium animalis ssp. lactis no. LMG P-28149 and being selected from the group consisting of constituents of the cell wall, of cell organites, nucleic acids, constituents of the cell membrane, and cell metabolites.

As an illustration and not as a limitation, the constituents of the cell wall are selected from the group consisting of peptidoglycans, proteins, polysaccharides, teichoic acid, or of a combination of the latter.

As an illustration and not as a limitation, the metabolites are selected from the group consisting of organic acids, inorganic acids, proteins, peptides, amino acids, enzymes, lipids, carbohydrates, glycolipids, glycoproteins, vitamins, salts, metals or a combination thereof.

In a particular embodiment, the composition according to the invention comprises butyrate, or at least one of its derivatives, and/or propionate, or at least one of its derivatives, the induction of said butyrate and/or of said propionate being promoted by Bifidobacterium animalis ssp. lactis no. LMG P-28149.

Optionally, the composition according to the invention comprises at least one additional probiotic, said additional probiotic being selected from the group consisting of the following probiotics: Archaea, Firmicutes, Bacteroidetes, Proteobacteria, Actinobacteria, Verrucomicrobia, Fusobacteria, Metanobacteria, Spirochaetes, Fibrobacters, Deferribacteres, Deinococcus, Thermus, Cyanobacteria, Methanobrevibacterium, Lactobacillus, Peptostreptococcus, Ruminococcus, Coprococcus, Subdolingranulum, Dorea, Bulleidia, Anaerofustis, Gemella, Roseburia, Catenibacterium, Dialister, Anaerotruncus, Staphylococcus, Micrococcus, Propionibacterium, Enterobacteriaceae (non-pathogenic), Faecalibacterium, Bacteroides, Parabacteroides, Prevotella, Eubacterium, Akkermansia, Bacillus, Butyrivibrio, and Clostridium, or a combination thereof.

Preferably, the composition according to the invention may further comprise at least one fungus and/or yeast strain, said strain being selected from the following group: Saccharomyces, Candida, Pichia, Debaryomyces, Torulopsis, Aspergillus, Rhizopus, Mucor, and Penicillium.

Preferably, the composition according to the invention comprises a carrier for encapsulating a probiotic in which said Bifidobacterium animalis ssp. lactis no. LMG P-28149 and optionally said at least one additional probiotic are encapsulated.

Advantageously, said fungus and/or said yeast are also encapsulated in said carrier.

Alternatively, the encapsulation carrier comprises at least one substance selected from the group consisting of alginate, chitosan, pectin, pullulan, gelatin, carrageenan, agar gel or a combination thereof.

Preferentially, said at least one substance is a hydrocolloid.

Advantageously, the composition according to the invention comprises at least one source of food selected from the group consisting of a monosaccharide, a polysaccharide, an amino acid, a peptide, a protein, a vitamin, a yeast extract, a halide salt, an alkaline or earth-alkaline metal, an antioxidant, glycerol, zinc acetate, zinc chloride, zinc lactate, ascorbic acid, citric acid, a plant oil, milk fat, or a combination thereof.

In a particular embodiment, the composition according to the invention is a symbiotic composition comprising at least one prebiotic.

Preferably, said at least one prebiotic is selected in a non-limiting way from the group consisting of oligosaccharides, fructo-oligosaccharides, galacto-oligosaccharides, xylo-oligosaccharides, inulin or its derivatives, lactulose or its derivatives, mannan-oligosaccharides, or a combination of the latter.

Preferentially, the composition according to the invention comprises a first enteric coating covering said Bifidobacterium animalis ssp. lactis no. LMG P-28149 and optionally said at least one additional probiotic. In particular, the first enteric coating is selected from the group consisting of ethyl cellulose, hydroxypropyl cellulose, carboxymethyl cellulose, polymers (such as for example Eudragit®), or a combination thereof.

Alternatively, the composition further comprises a second external coating selected from the group consisting of alginate, chitosan, pectin, pullulan, gelatin, carrageenan, agar gel, cellulose, hemicellulose, ethylcellulose, carboxymethylcellulose, or a combination thereof.

In another optional embodiment, the composition according to the invention comprises one or several biocompatible excipients.

The composition according to the invention may be intended for curative or preventive treatment of excessive body weight gain (i.e. widely exceeding the weight gain which accompanies pregnancy) in pregnant women or in women who have given birth or in men in a “couvade” ritual or having been in a “couvade” ritual.

According to the invention, the composition may appear as a food composition, of a food based on the composition or further of a food supplement comprising Bifidobacterium animalis ssp. lactis no. LMG P-28149, preferably in the living condition.

By the terms of “food composition”, in the sense of the present invention should be meant a composition which one commonly finds on the food market (for example, snacks, prepared dishes, beverages, etc.).

By the terms of “food supplement”, should be understood in the sense of the present invention a foodstuff, the purpose of which is to provide a supplement of nutrients or substances having a nutritional and/or physiological effect.

According to the invention, the composition may be provided for use and intake via an oral, or sublingual route, but also via a respiratory, preferably nasal or bronchial, or further rectal.

The composition may also be an injectable liquid composition based at least on an element derived from the probiotic and intended to be injected subcutaneously.

Preferentially, the composition according to the invention may be conditioned as tablets, globules, gelatin capsules, granules, powders, fluids, liquids, creams or sprays.

According to the invention, the composition may be used for curative or preventive treatment of excessive body weight gain (i.e. widely exceeding the weight gain which accompanies pregnancy) in pregnant women or who have given birth or in men in a “couvade” ritual or having been in a “couvade” ritual.

Other embodiments and uses of the therapeutic composition according to the invention are given in the appended claims.

The present invention also relates to a non-therapeutic cosmetic use of a composition comprising at least a probiotic, in overweight human beings or having been overweight, said probiotic being Bifidobacterium animalis ssp. lactis no. LMG P-28149.

Preferably, said composition for a non-therapeutic cosmetic use comprises at least one additional probiotic selected from the group consisting of the following probiotics: Archaea, Firmicutes, Bacteroidetes, Proteobacteria, Actinobacteria, Verrucomicrobia, Fusobacteria, Metanobacteria, Spirochaetes, Fibrobacters, Deferribacteres, Deinococcus, Thermus, Cyanobacteria, Methanobrevibacterium, Lactobacillus, Peptostreptococcus, Ruminococcus, Coprococcus, Subdolingranulum, Dorea, Bulleidia, Anaerofustis, Gemella, Roseburia, Catenibacterium, Dialister, Anaerotruncus, Staphylococcus, Micrococcus, Propionibacterium, Enterobacteriaceae (non-pathogenic), Faecalibacterium, Bacteroides, Parabacteroides, Prevotella, Eubacterium, Akkermansia, Bacillus, Butyrivibrio, and Clostridium, or a combination thereof.

Advantageously, said composition for a non-therapeutic cosmetic use comprises a fungus and/or yeast strain selected from the group consisting of Saccharomyces, Candida, Pichia, Debaryomyces, Torulopsis, Aspergillus, Rhizopus, Mucor, and Penicillium.

Preferentially, in said composition for a non-therapeutic cosmetic use, said Bifidobacterium animalis ssp. lactis no. LMG P-28149 and optionally said at least one additional probiotic are encapsulated in an encapsulation carrier.

Advantageously, in said composition for a non-therapeutic cosmetic use, said encapsulation carrier comprises at least one substance selected from the group consisting of alginate, chitosan, pectin, pullulan, gelatin, carrageenan, agar gel and a combination thereof.

Advantageously, said composition for a non-therapeutic cosmetic use comprises at least one source of food selected from the group consisting of a monosaccharide, polysaccharide, amino acid, peptide, protein, vitamin, yeast extract, halide salt of an alkaline or earth-alkaline metal, antioxidant, glycerol, zinc acetate, zinc chloride, zinc lactate, ascorbic acid, citric acid, plant oil, milk fat, or a combination thereof.

Preferably, said composition for a non-therapeutic cosmetic use further comprises at least one prebiotic, thereby forming a symbiotic composition.

Preferably, said composition for a non-therapeutic cosmetic use comprises a first enteric coating covering said Bifidobacterium animalis ssp. lactis no. LMG P-28149 and optionally said at least one additional probiotic.

Advantageously, in said composition for a non-therapeutic cosmetic use, said first enteric coating is selected from the group consisting of ethylcellulose, hydroxypropylcellulose, carboxymethyl cellulose, Eudragit®, or a combination thereof.

Preferably, said composition for a non-therapeutic cosmetic use comprises a second external coating selected from the group consisting of alginate, chitosan, pectin, pullulan, gelatin, carrageenan, agar gel, cellulose, hemicellulose, ethylcellulose, carboxymethylcellulose, or a combination thereof.

Advantageously, said composition for a non-therapeutic cosmetic use further comprises one or several biocompatible excipients.

Other forms for non-therapeutic cosmetic use of the composition according to the invention are indicated in the appended claims.

The present invention also relates to a culture medium of the probiotic Bifidobacterium animalis ssp. lactis no. LMG P-28149 comprising at least one protein source and at least one source of carbohydrates, said culture medium being characterized in that it further comprises glutathion.

Glutathion is a tripeptide involved in the maintaining of the redox potential of the cytoplasm of the cells and in a certain number of detoxification and removal reactions of reactive species of oxygen.

Surprisingly, it was shown, within the scope of the present invention, that such a culture medium comprising glutathion optimizes the efficiency of the strain no. LMG P-28149 of the probiotic Bifidobacterium animalis ssp. lactis, for which a curative or preventive action was observed on the body weight gain in overweight human beings or having been overweight.

Preferably, the culture medium according to the invention comprises glutathion in a concentration comprised between 20 and 30 g/l of culture medium.

Advantageously, according to the invention, said protein source of said culture medium is selected from the group consisting of a lactoserum peptone, a casein peptone, a plant or bacterial peptone, and of a combination thereof.

Preferably, according to the invention, said source of carbohydrates of said culture medium comprises at least one sugar or a mixture of sugars, which are selected from the group formed by lactose, glucose, galactose, fructose, maltodextrin, starch, trehalose, maltotriose, and a combination thereof.

Preferably, according to the invention, said culture medium further comprises at least one amino-sugar, for example glucosamine or galactosamine.

Preferably, according to the invention, said culture medium further comprises at least one yeast extract.

Preferably, according to the invention, said culture medium further comprises at least one egg extract.

Other embodiments of the culture medium according to the invention are indicated in the appended claims.

The present invention also relates to a method for manufacturing by fermentation of the probiotic Bifidobacterium animalis ssp. lactis no. LMG P-28149, said method comprising at least one step for cultivating said probiotic in a culture medium according to the invention.

Other embodiments of the method according to the invention are indicated in the appended claims.

Other features and advantages of the invention will become apparent from the description given hereafter, in a non-limiting way and with reference to the examples described below.

These examples repeat results obtained in mice which were completed with results obtained in vitro from immune cells isolated from human blood.

Although the results of the tests repeated in Examples 1 to 4 below were obtained in mice, it is understood that similar results are expected in human beings.

In particular, an effect specific to the strain on the body weight gain in mammals, in particular in rodents was demonstrated within the scope of the present invention.

Indeed, administering the strain LMG P-28149 of the probiotic Bifidobacterium animalis ssp. Lactis, alone or in combination with at least one other probiotic, induces a reduction in the body weight gain together with improvement of the inflammatory and metabolic parameters in mammals, in particular in rodents, overweight, including insulin resistance.

In particular, metabolic protective effects of the composition based on the probiotic strain according to the invention were demonstrated within the scope of the present invention, which are moreover associated with restoration, within adipose tissues, of the expression of the PPARγ and of cell remodeling, and of a protective effect against liver steatosis.

The composition based on the strain according to the invention also allows modulation of the expression of the receptors related to the transport of fatty acids, and in particular restoration of the receptors coupled with the G protein (GPR41 and GPR43) involved in the transport of short chain fatty acids (SOFA) which are important actors in the satiation mechanism induced by the absorption of nutrients.

Indeed, the positive action of the strain according to the invention on the size of the adipocytes and on the production of pro-inflammatory cytokines and chemokines (MCP-1, IL-6, TNF-alpha, etc.) was demonstrated, some of them being directly at the origin of insulin resistance.

It was observed that the strain according to the invention moreover allows re-establishment of the lipid profile and of the metabolism of glucose in mammals, preventing the risk of developing diseases directly related to the metabolic syndrome like dyslipidemia (and therefore diabetes of type II) and hyperglycemia.

Finally, the link between the taking of the probiotic strain according to the invention and the re-colonization of the intestinal tract by Akkermansia, a bacterium notorious for its role in the prevention of obesity in human beings and strongly reduced in overweight or obese individuals was demonstrated.

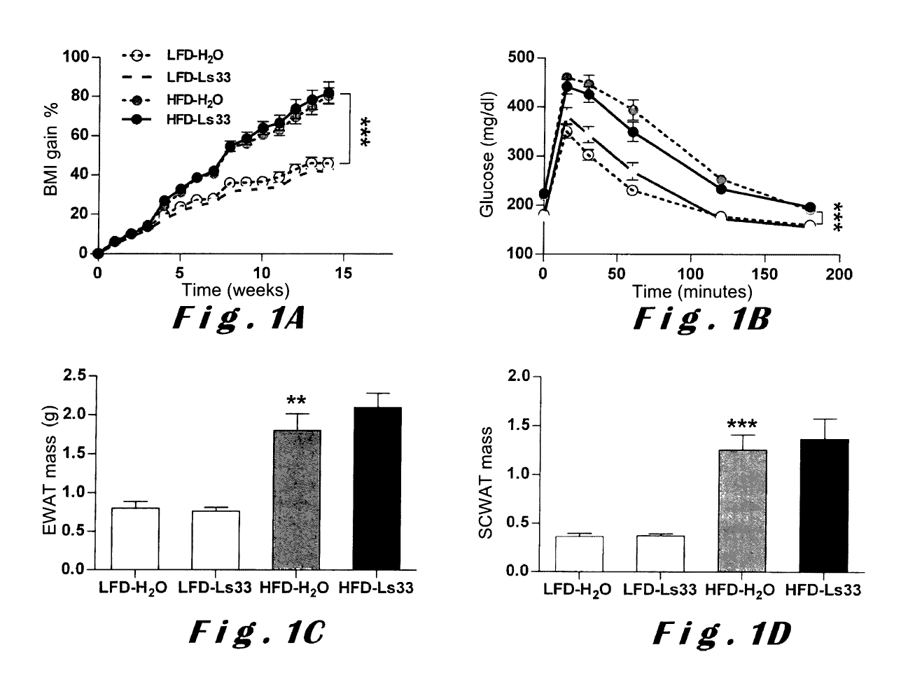

FIG. 1 illustrates, in mice during development of overweight and then of obesity, the action of Ls33 on (A) the time-dependent change in the body weight gain (in %), (B) tolerance to glucose (GT), (C) the weight (mass) of the epididymal adipose tissue

(EWAT), and (D) the weight (mass) of the subcutaneous adipose tissue (SCWAT). The data are expressed as an average (from 10 to 15 mice per group)±standard-deviation from the mean (SEM). **p<0.01; ***p<0.001. * corresponds to the comparison between the fat-enriched diet (HFD) vs the control diet (LFD) under identical interventional treatment (measurement of the diet effect).

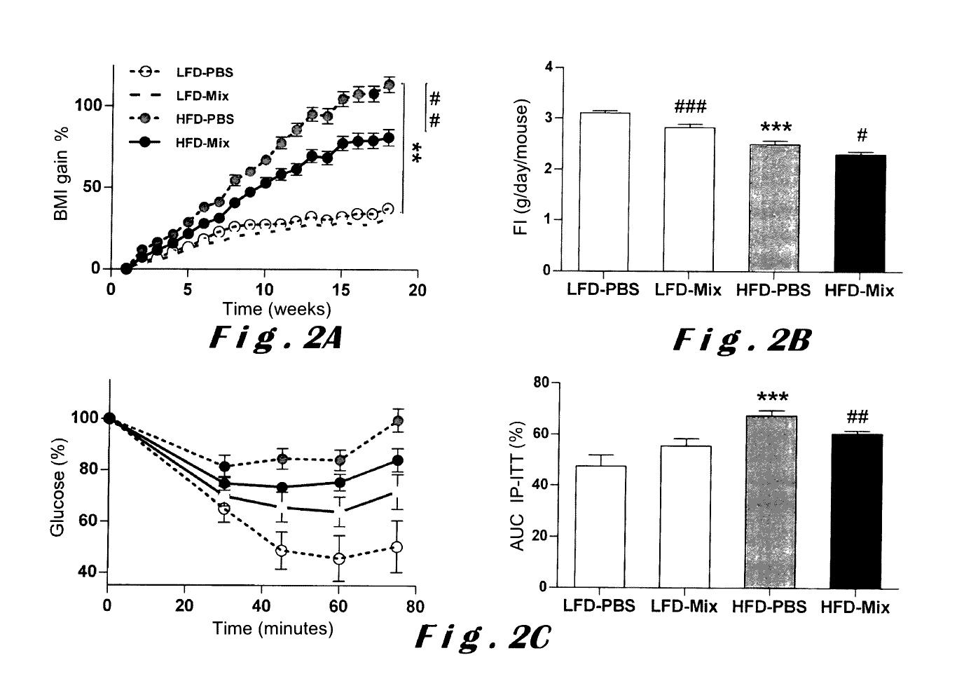

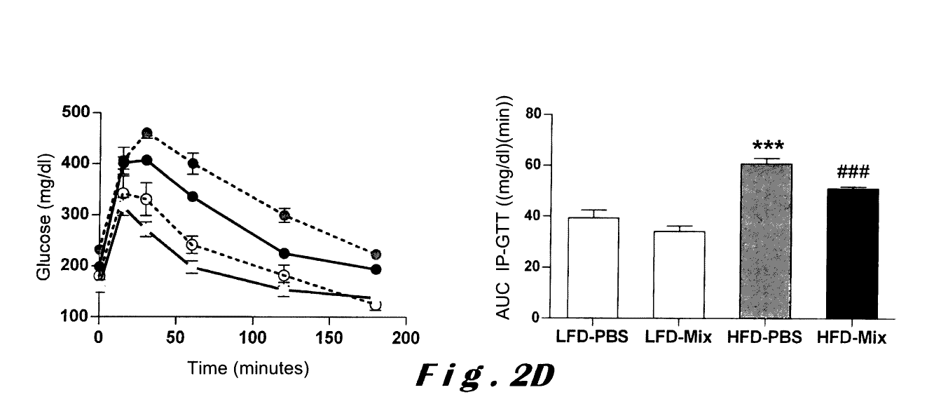

FIG. 2 illustrates in mice during development of overweight and then of obesity, the action of the Mix on (A) the time-dependent change in the body weight gain (in %), (B) the accumulated intake of food (in gram/day/mouse), (C) tolerance to insulin (IT), and (D) the GT. The data are expressed as a mean (for 5 to 14 mice per group)±standard-deviation from the mean (SEM). **p<0.01; ***p<0.001; ^{#}p<0.05; ^{##}p>0.01; ^{###}p<0.001. * corresponds to the comparison between the HFD diet vs the LFD diet with identical interventional treatment (measurement of the effect of the diet). ^{#} corresponds to the comparison between the Mix vs the saline phosphate buffer (PBS) at an identical diet (measurement of the effect of the probiotic or mixture of probiotics according to the invention).

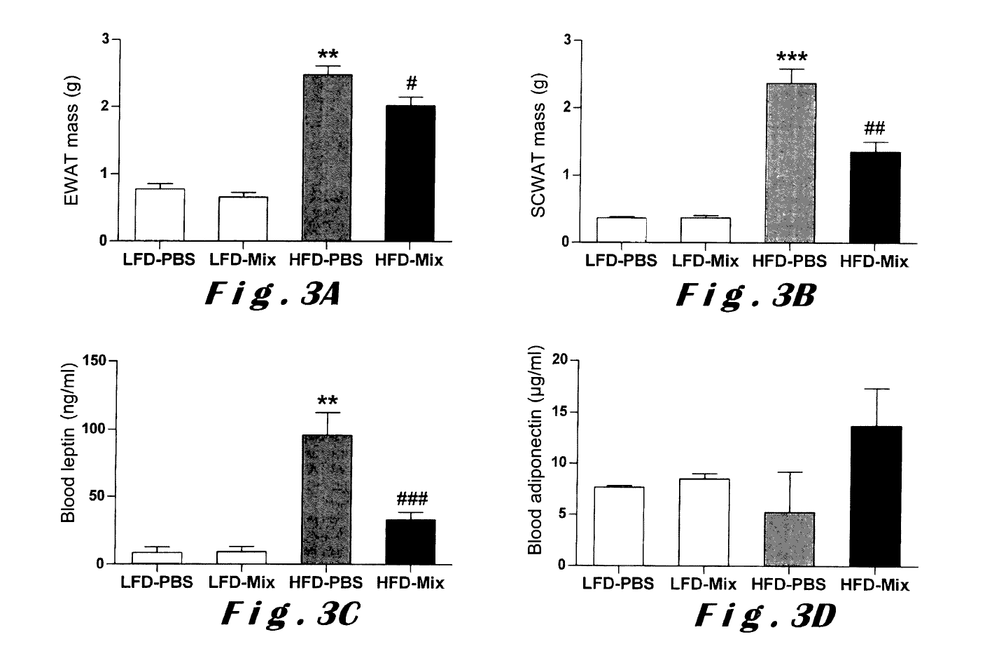

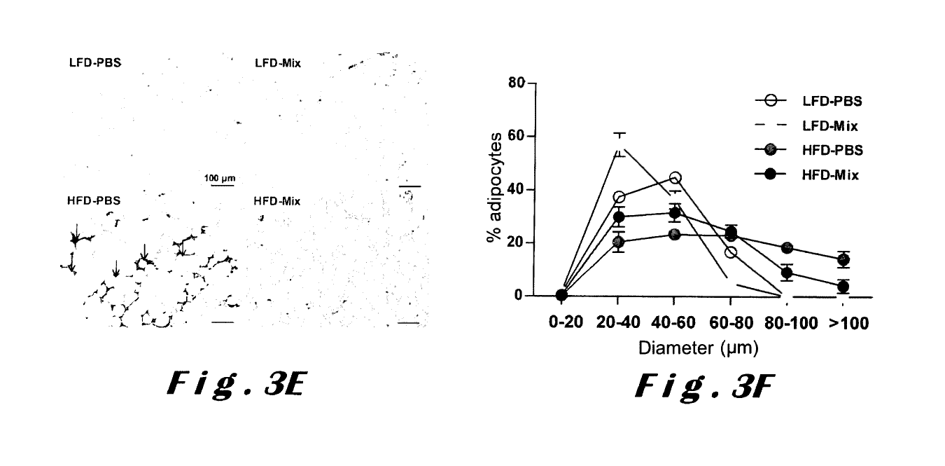

FIG. 3 illustrates in mice during overweight and then obesity development, the action of the Mix on (A) the weight (mass) of EWAT, (B) the weight (mass) of SCWAT, (C) the amount of leptin in the blood (ng/ml), (D) the amount of adiponectin in the blood (□g/ml), (E) the histology of EWAT (representative cuts of each of the experimental groups), and (F) the size distribution of the adipocytes in EWATs. The data are expressed as a mean (10 to 15 mice per group)±standard-deviation from the mean (SEM). **p<0.01; ***p<0.001; ^{#}p<0.05; ^{##}p<0.01; ^{###}p<0.001. *corresponds to the comparison between the HFD diet vs the LFD diet with identical interventional treatment (measurement of the effect of the diet). ^{#} corresponds to the comparison between the Mix vs PBS at an identical diet (measurement of the effect of the probiotic or mixture of probiotics according to the invention).

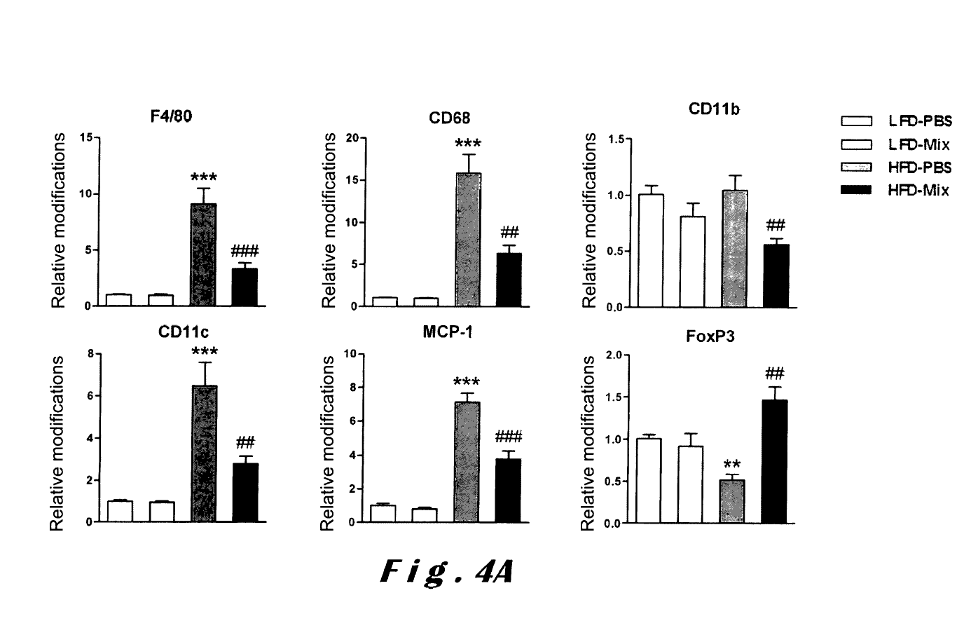

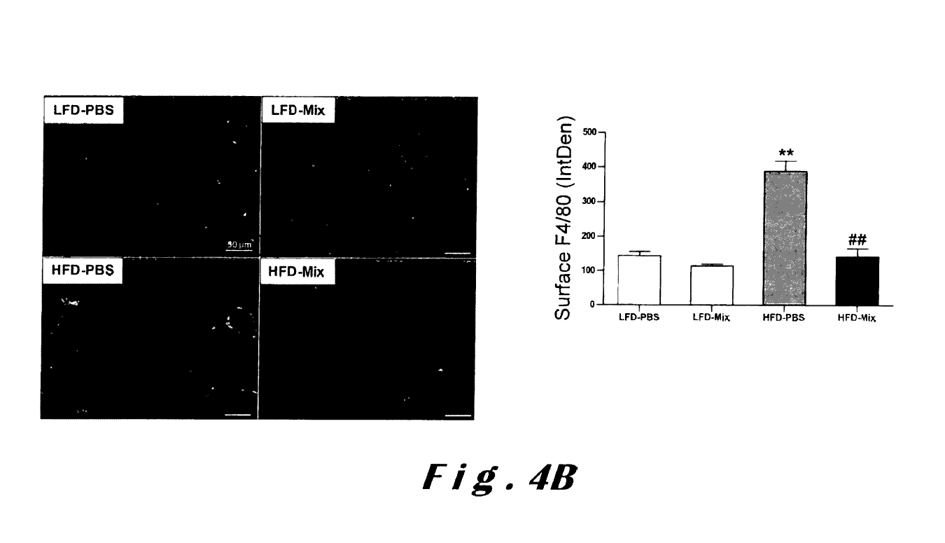

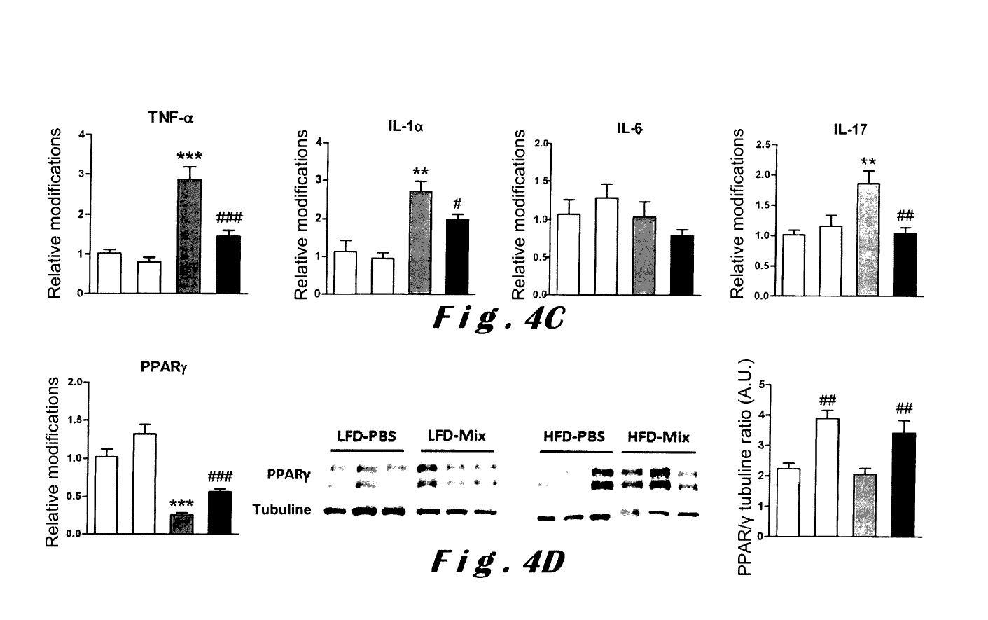

FIG. 4 illustrates in mice during development of overweight and then of obesity, the action of the Mix on (A) the cell composition of the EWATs (analysis of expression of specific genes of the monocytes/macrophages (F4/80, CD68, CD11b, CD11c), of the regulating T lymphocytes (FoxP3) and of the chemokine MCP-1), (B) the presence of macrophages in the EWATs (immunofluorescent marking specific to F4/80 and quantification in terms of integrated density [IntDen]), (C) inflammation of the EWATs (analysis of expression of genes of pro-inflammatory cytokines Tnfα, IL-1a, IL-6 and IL-17), and (D) expression of PPAR gamma (PPARγ), at the messenger RNA (left, gene expression) and of the protein (center left, Western-blot and quantification [in arbitrary unit: A.U.]). The data are expressed as a mean (from 5 to 14 mice per group)±standard-deviation from the mean (SEM). **p<0.01; ***p<0.001; ^{##}p<0.01; ^{###}p<0.001. *corresponds to the comparison between the HFD diet vs the LFD diet with identical interventional treatment (measurement of the effect of the diet). ^{#}corresponds to the comparison between the Mix vs PBS at an identical diet (measurement of the effect of the probiotic or of the mixture of probiotics.

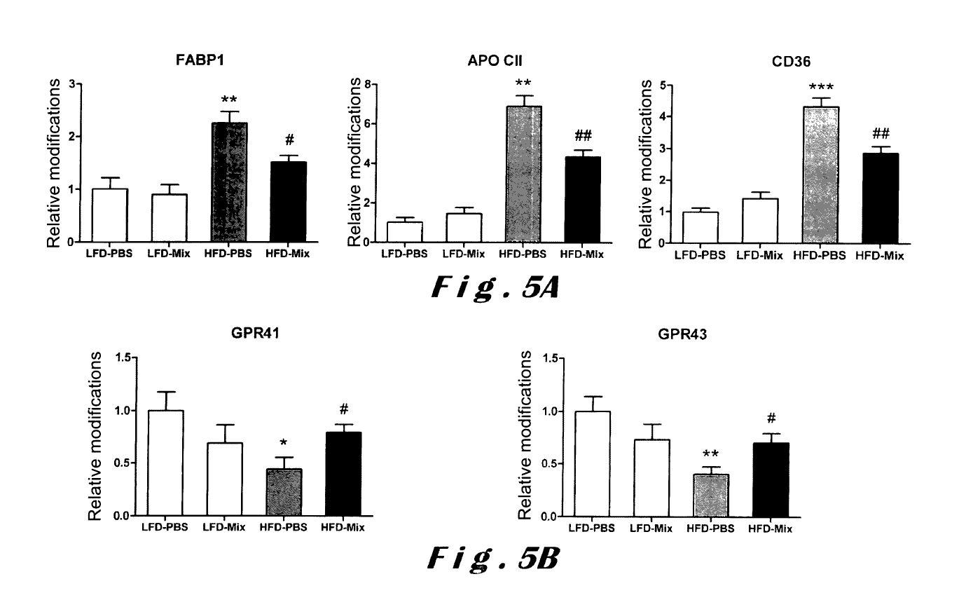

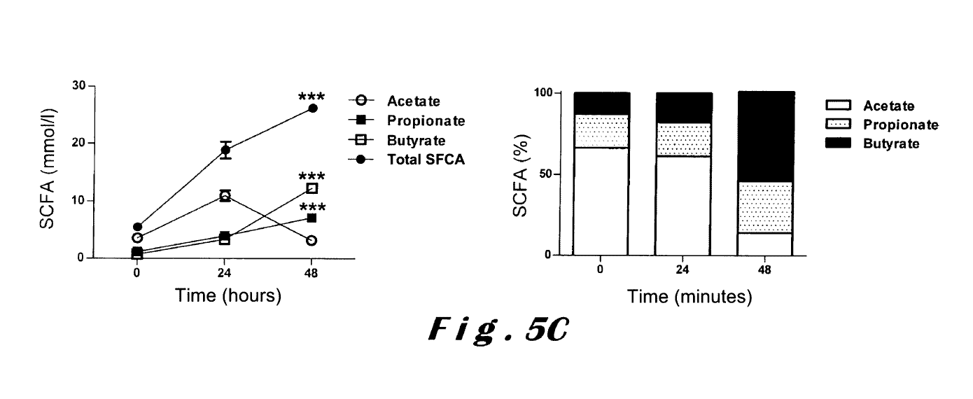

FIG. 5 illustrates in mice during development of overweight and then of obesity, the action of the Mix on (A) factors implied in the metabolism of lipids (analysis of the expression of the genes FABP1, APO CII, and CD36), (B) receptors to short chain fatty acids (SOFA), by analysis of expression of the genes GPR41 and GPR43). The data are expressed as a mean (from 5 to 14 mice per group)±standard-deviation from the mean (SEM). **p<0.01; ***p<0.001; ^{##}p<0.01; ^{###}p<0.001. *corresponds to the comparison between the HFD (high fat diet) vs the LFD (low fat diet) diet with identical interventional treatment (measurement of the effect of the diet). ^{#} corresponds to the comparison between the Mix vs PBS at an identical diet (measurement of the effect of the probiotic or mixture of probiotics).

FIG. 5(C) illustrates the production level of the total SCFAs (left) and proportional levels of acetate, butyrate and propionate (right) after 24 h and 48 h of incubation with the probiotic mixture Mix in the model simulating the intestinal ecosystem (SHIME), in vitro. *** compared with time 0.

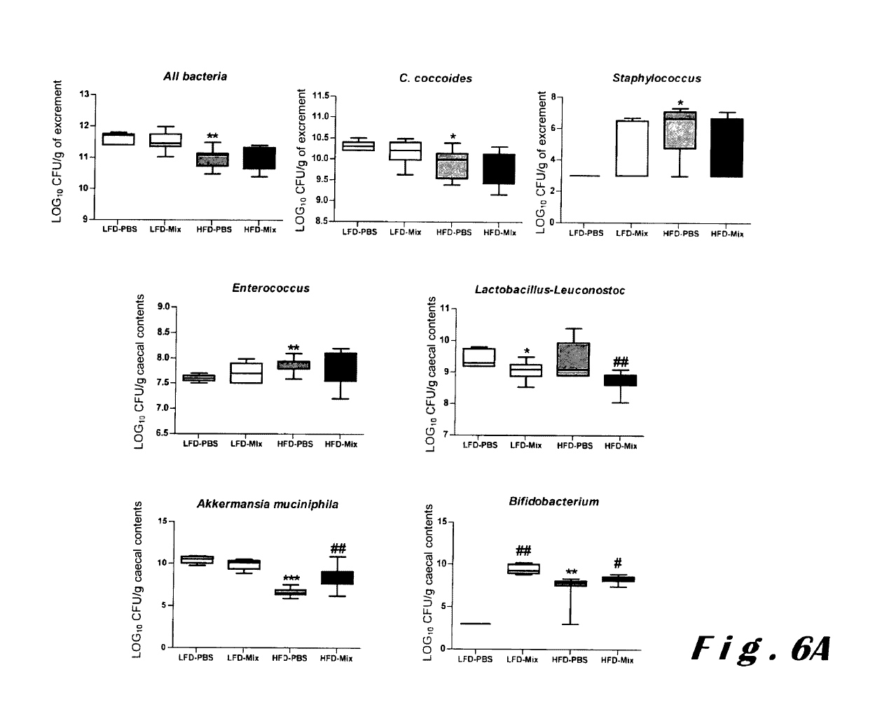

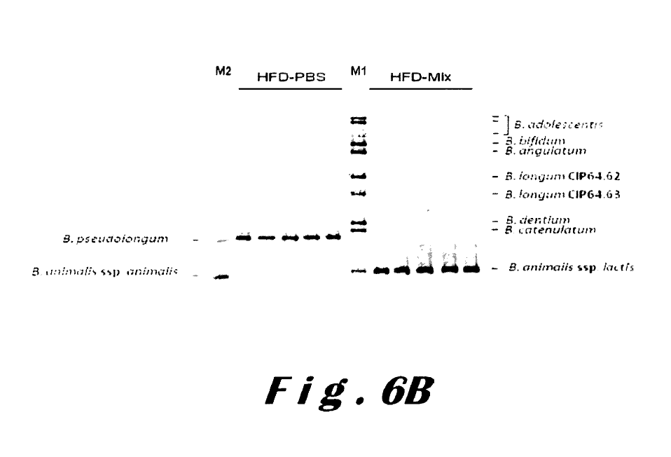

FIG. 6 illustrates in mice during development of overweight and obesity, the effect of the Mix on (A) the composition of the intestinal microbiot and in particular on the population of Bifidobacteria and Akkermansia muciniphila. (B) The detected species of Bifidobacterium in HFD mice either treated or not with the mix was determined by TGGE analysis for 5 mice, representative of the HFD-PBS and HFD-Mix groups. The markers M1 or M2 correspond to the mixtures of the indicated strains. *p<0.05; **p<0.01; ^{#}p<0.05; ^{##}p<0.01. *corresponds to the comparison between the HFD diet vs the LFD diet with identical interventional treatment (measurement of the effect of the diet). ^{#} corresponds to the comparison between the Mix vs PBS at an identical diet (measurement of the effect of the probiotic or of the mixture of probiotics according to the invention).

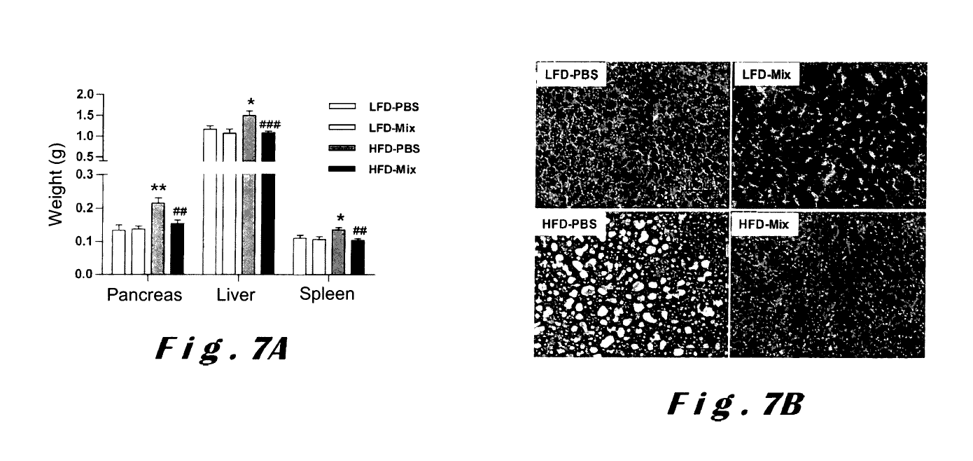

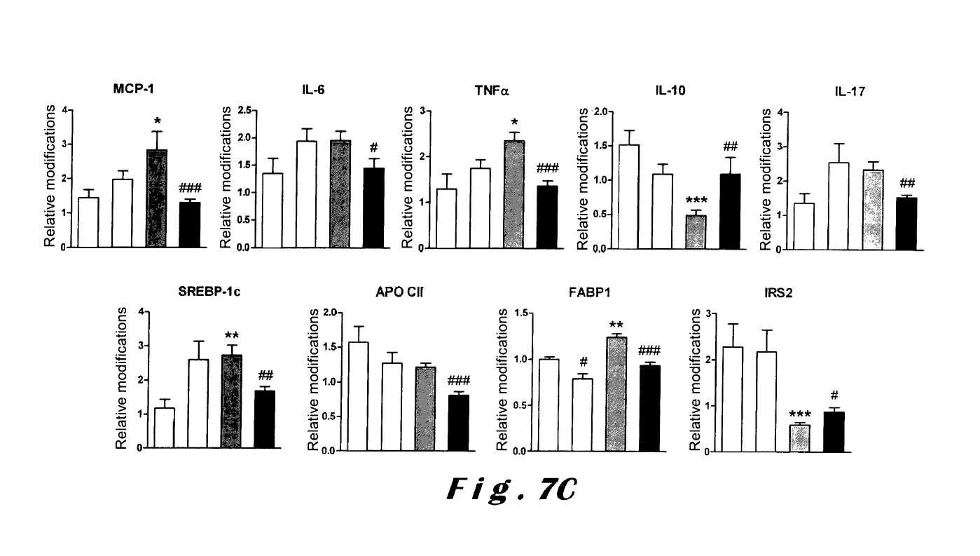

FIG. 7 illustrates in mice during development of overweight and then of obesity, the action of the Mix on (A) the weight (mass) of the pancreas, of the liver and of the spleen, (B) the accumulation of lipid droplets (steatosis, see the histological cuts representative of each of the experimental groups), and (C) different markers of the inflammation or implied in the lipid metabolism or the response to insulin (analysis of expression of the genes mcp-1, IL-6, TNFα, IL-10, IL-17, srebp-1c, APOCII Fabp1 and IRS2). The data are expressed as a mean (from 5 to 14 mice per group)±standard-deviation from the mean (SEM). **p<0.01; ***p<0.001; ^{##}p<0.01; ^{###}p<0.001; ^{####}p>0.0001. * corresponds to the comparison between the HFD diet vs the LFD diet with identical interventional treatment (measurement of the effect of the diet). ^{#} corresponds to the comparison between the Mix vs PBS at an identical diet (measurement of the effect of the probiotic or of the mixture of probiotics according to the invention).

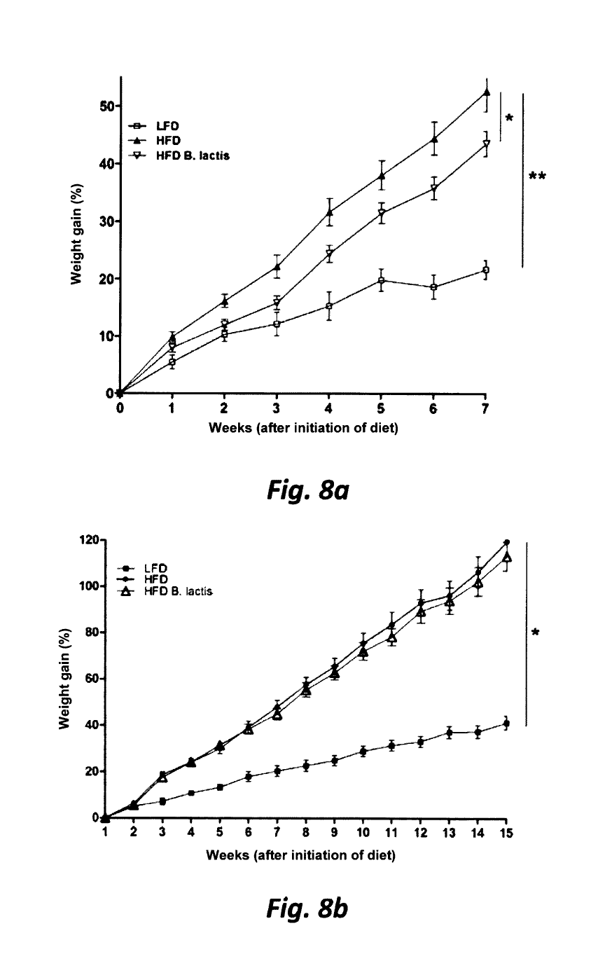

FIGS. 8a and 8b illustrate the impact of the probiotic Bifidobacterium animalis ssp. lactis no. LMG P-28149 according to the invention on the weight gain over time depending on whether the probiotic was cultivated (FIG. 8a) or not cultivated (FIG. 8b) in a culture medium according to the invention. LFD=low fat diet; HFD=high fat diet; HFD B. lactis=high fat diet associated with taking the composition according to the invention when the probiotic is cultivated in the presence of glutathion (FIG. 8a) or in the absence of glutathion (FIG. 8b).

The following procedure was observed for harvesting the results in vivo discussed in the Examples 1 to 4 which follow.

Procedures In Vivo

Mice, Bacteria Strains, and Diets

The tests were conducted on male mice C57BL/6J, 5 weeks old at the beginning of the experimentation.

The strain Ls33 of Lactobacillus L. salivarius was provided by Danisco (Madison, Wis., USA).

The mixture of probiotics (referenced as Mix in the following examples) comprises two different strains: one strain of L. rhamnosus LMG S-28148 and a strain of Bifidobacterium animalis ssp. lactis no. LMG P-28149 in a CFU (colony-forming units) ratio of 1:1 (total 10⁹ CFU). The diets imposed to the mice are divided in a first diet with a low fat content (LFD: D12450B; 10% kcal from fats) and in a second diet with a high fat level (HFD: D12492, 60% kcal coming from the fats). The diets are provided by the company Research Diet.

Treatment Procedure of the Animals

For 5 consecutive days per week, each mouse received oral administration of 30 μL of Ls33-10⁹ CFU (colony-forming units or the number of colony-forming units present in the oral composition) in sterilized water (H₂O) (group Ls33-H₂O), of sterilized water (group H₂O), or of a mixture of probiotics (group Mix; 5 10⁸ CFU for each strain) in the saline phosphate buffer (PBS) (group Mix-PBS or group PBS).

After a week of treatment, the mice treated with Ls33, with water, with Mix, or with PBS were randomly assigned to an LFD diet (n=5 per group) or HFD diet (n=15 per group). The body weight and the food intake were recorded weekly.

When the animals were sacrificed, the blood, the epididymal white adipose tissues (EWATs) and subcutaneous white adipose tissues (SCWATs), the liver, the spleen, the small intestine and the pancreas were collected.

Tests of Tolerance to Insulin and to Glucose

The glucose tolerance (GTT) and insulin tolerance (ITT) tests were conducted after, respectively 12 and 14 weeks of diet. The animals were fasted for a period of 6 hours before receiving an intra-peritoneal administration (IP) of glucose (D-glucose, 1 g/kg of body weight) (GTT) or of insulin (0.75 UI/kg of body weight) (ITT). The glucose levels in the blood were measured with an automatic glucometer available commercially (for example, an ACCU-CHEK® performa) on a blood sample taken at the tail before injecting glucose (GTT) or insulin (ITT) and at different times after injection of glucose (GTT) or of insulin (ITT).

Blood Analyses

The plasma levels of leptin, adiponectin, MCP-1, and insulin were measured by using marketed ELISA kits. The levels of non-esterified fatty acids (NEFA), of triglycerides, of glycerol, of HDL cholesterol, and LDL cholesterol were determined by using analyses kits available on the market (for example, the Abcam kit developed by Cambridge, GB).

Histological Analyses and Immuno-Histochemical Analyses

The samples of liver and of white adipose tissues (EWAT) were set in a solution of paraformaldehyde at 4%, then included in paraffin and finally cut, deposited on a slide and then stained with hematoxylin eosin (H&E). The morphometric analysis of the white adipose tissues (EWAT), at least 10 fields (which represents about 100 adipocytes) per section was carried out by using the imaging program Image J (NIH image, National Center for Biotechnology Information).

Analyses of the Expression of the Genes

The total RNA of the white adipose tissues of liver and intestine was extracted (according to the method known to one skilled in the art) so as to be then back-transcripted (an amount of 1 μg was re-transcripted for each of the aforementioned tissue categories). Quantitative real time PCR (POLYMERASE CHAIN REACTION) (RT-qPCR) was carried out according to a procedure known to one skilled in the art.

Statistical Analyses

The data are expressed as the mean±standard-deviation relatively to the mean (SEM). Statistical analyses were carried out by using the Kruskal Wallis test followed by the Mann-Whitney U test. The differences between the experimental groups are considered as being statistically significant when the value p is less than 0.05.

The following procedure was observed for harvesting the in vitro results discussed in Examples 5 and 6.

Procedure In Vitro

Simulator of the Human Microbial Ecosystem—SHIME Model

The SHIME reactor which simulates the human gastro-intestinal tract was set into place according to a procedure well known to one skilled in the art.

The Mix was added into the reactor and the short chain fatty acid productions (SOFA) were measured at times t=0, t=24 h, and t=48 h of incubation at 37° C. under anaerobic atmosphere.

Evaluation of the Anti-Inflammatory Effect of the Strain Bifidobacterium animalis ssp. lactis no. LMG P-28149 on Human Blood Cells

Fresh human blood, obtained from four healthy donors, was diluted in a ratio 1:1 with PBS-Ca (GIBCO), deposited on a layer of Ficoll (GIBCO) and centrifuged at 400 G for 30 minutes at 20° C.

The mononuclear cells of peripheral blood (PBMC) were isolated from blood after centrifugation and suspended in a final volume of 50 ml by using PBS-Ca, for them being washed three times at 750 G for 10 minutes at 20° C. The PBMCs were then re-suspended in an RPMI medium (GIBCO), completed with 10% (v/v) of decomplemented calf serum (serum brought to 56° C. for 30 minutes), 1% (w/v) of L-glutamine (GIBCO), and of gentamycin (30 μg/ml) (GIBCO). The PBMCs were counted and their number adjusted to 2×10⁶ cells per ml. The preparation is distributed (1 ml) in 24-well culture plates.

### EXAMPLE 1

### Ls33 vs Mix

This example illustrates the impact of Ls33 on the development of overweight and obesity in mice.

FIG. 1 illustrates, for a cure of 15 weeks in Ls33 or water and a food diet with a low fat level (LFD) or a high fat level (HFD), the following indicators:

(A) the time-dependent change in the body weight gain, expressed as a percentage relatively to the weight measured on day d=0;

(B) the glucose tolerance test (GTT) conducted after 12 weeks of diet. The glucose levels (in mg/dl) were measured in mice, directly after a fasting period of 6 hours, at the times t (in mins) indicated on the graph, after intra-peritoneal injection (IP) of glucose (which corresponds to time t=0).

(C) the weight (mass) of the epididymal adipose tissue (EWAT) (in g) after 15 weeks of diet (weighed during the sacrifice);

(D) the weight (mass) of the subcutaneous adipose tissue (SCWAT) (in g) after 15 weeks of diet (weighed during the sacrifice).

As shown by the results exhibited on this figure, the strain Ls33, in spite of its demonstrated anti-inflammatory activity in vitro and in other pathological models (i.e. intestinal inflammations), does not show any (positive or negative) effect on the various indicators A to D.

Conversely, the administration of the mixture Mix shows significant protective effects. These results are illustrated in FIG. 2.

This figure illustrates, after 17 weeks of treatment with the mixture of probiotics (Mix) or PBS and for a diet with a low level of fats (LFD) or a high level of fats (HFD), the corresponding results:

(A) to the time-dependent change in the body weight gain (expressed as a percentage relatively to the measured weight on day d=0);

(B) to the accumulated food intake per mouse and per day (g/day/mouse);

(C) to the insulin tolerance test (ITT) conducted 14 weeks after starting the diet: the glucose levels in the blood were measured after intraperitoneal injection (IP) of insulin. The results show that the normalized levels (in % relatively to the measured glucose levels before injection)±SEM and the means±SEM of the values of the area under the curve (AUC) for each normalized glucose curve after injection of insulin;

(D) to the glucose tolerance test (GTT) conducted after 12 weeks of diet. The glucose levels (in mg/dl) were measured in mice after IP injection of glucose and the AUC values were calculated.

For the group of HFD-Mix mice, a less significant body weight gain was observed (gain of 80.97%±4.96%) than that obtained in the HFD-PBS group (gain of 113.51%±4.89%) (see FIG. 2A).

As shown by FIG. 2B, the mice of the HFD group treated with mixture of probiotics Mix have a reduction of the accumulated food intake (Food intake, FI). Interestingly, the beneficial effects observed on the body weight gain and the FI index appeared rapidly after the beginning of the treatment (from the fourth week).

The administration of the Mix mixture improves homeostasia of glucose for the HFD group as shown by the lesser glucose and insulin levels from the table 1 below:

TABLE 1

| | Groups | | | |
|---|---|---|---|---|
| Indicator | LFD-PBS | LFD-Mix | HFD-PBS | HFD-Mix |
| Glucose (mg/dl) | 180.4 ± 30.88 | 160.3 ± 28.96 | 231.67 ± 21.7* | 198.43 ± 15^{##} |
| Insulin(ng/ml) | 0.72 ± 0.37 | 0.98 ± 0.24 | 2.96 ± 0.57* | 1.57 ± 0.23^{#} |
| NEFA (mmol/l) | 0.85 ± 0.06 | 1.07 ± 0.07 | 1.06 ± 0.03 | 0.9 ± 0.08 |
| Glycerol (mmol/l) | 0.13 ± 0.06 | 0.24 ± 0.05 | 0.38 ± 0.1 | 0.34 ± 0.05 |
| Triglyceride(mmol/l) | 0.4 ± 0.009 | 0.46 ± 0.02 | 0.41 ± 0.03 | 0.41 ± 0.03 |
| Cholesterol mmol/l) | 1.35 ± 0.05 | 1.33 ± 0.05 | 1.94 ± 0.04*** | 1.72 ± 0.3^{###} |
| HDL (mmol/l) | 1.09 ± 0.04 | 1.07 ± 0.04 | 1.5 ± 0.01*** | 1.35 ± 0.02^{###} |
| LDL (mmol/l) | 0.25 ± 0.01 | 0.26 ± 0.01 | 0.45 ± 0.04*** | 0.36 ± 0.02^{###} |

The data of this table are expressed in mean ± standard-deviation relative to the mean (SEM) (corresponding to 5 and 14 mice per group).
*p < 0.05;
***p < 0.001;
^{#}p < 0.05;
^{##}p < 0.01;
^{###}p < 0.001.
*corresponds to the comparison between the HFD diet vs the LFD diet with identical interventional treatment (measurement of the effect of the diet).
^{#}corresponds to the comparison between the Mix vs PBS at an identical diet (measurement of the effect of the probiotic or of the mixture of probiotics).

Moreover, as shown by table 1, if the effects of the Mix remain limited to the NEFA, glycerol and triglyceride levels, it demonstrates a hypocholesterolemic action when it is associated with a decrease of the total cholesterol level and of the HDL cholesterol.

Further, the HOMA-IR index ((fasting insulin/ fasting glucose level)/22.5 level) is significantly reduced in mice of the HDF-Mix group, as compared with the control group (32.04±5.86 vs 81.57±19.63; p<0.01), which indicates improved insulin sensitivity, as confirmed by the results of the insulin tolerance test (see FIG. 2C).

Also, the mice treated with Mix are less intolerant to injections of glucose (see FIG. 2D).

### EXAMPLE 2

### Action of the Mix on the Inflammation of White Adipose Tissues

FIG. 3 illustrates the effects of the Mix on:

(A) the weight (mass) of the EWAT (in g);

(B) the weight (mass) of SCWAT (in g);

(C) the amounts of leptin in blood (in ng/ml) as measured by the ELISA method (an immuno-enzymatic assay method) in the serum of mice having fasted beforehand for 6 hours;

(D) the amounts of adiponectin in the blood (in pg/ml) measured by the ELISA method in the serum of mice having fasted beforehand for 6 hours;

(E) the histology of the epididymal adipose tissues (presentation of cuts stained with hematoxylin and with eosin representative of each of the experimental groups). The scale is 100 μm. The black arrows indicate the presence of cell infiltration; and

(F) the distribution of the size of the adipocytes of the EWAT. The results are shown in % of adipocytes per size class (0-20 μm, 20-40, etc.), while FIG. 7 illustrates the action of the Mix on:

(A) the weight (mass) of the pancreas, of the liver and of the spleen (in g);

(B) the accumulation of lipid droplets (i.e. steatosis) in the liver (presentation of cuts, stained with H&E, representative of each of the experimental groups). The scale is 100 μm; and

(C) the expression of genes coding for the pro- and anti-inflammatory cytokines and the receptors involved in the transport and metabolism of lipids, in the liver.

In comparison with the control groups HFD, the HFD-Mix group has a marked reduction of the adipose masses EWAT and SCWAT (see FIGS. 3A and 3B).

A marked reduction of the weights (masses) of the pancreas, of the liver and of the spleen is also observed in individuals of the HFD-Mix group (see FIG. 7A).

Further, while the mice of the controlled HFD group developed hepatic steatosis, the administration of the mixture of probiotics Mix gives the possibility of limiting the presence of lipid droplets in the tissues (see FIG. 7B).

In accordance with the decrease in the mass of white adipose tissues, the blood leptin levels prove to be significantly lower in the HFD-Mix group (see FIG. 3C), while the adiponectin levels tend to be higher (see FIG. 3D).

The histology of the epididymal adipose tissues showed a higher density of small adipocytes in the mice of group HFD-Mix (see FIGS. 3E and 3F).

It should be noted that if in the mice of the control group, the adipose tissues are infiltrated in a marked way by cells which surround the adipocytes (marked by an arrow in FIG. 3E), the tissue samples taken in mice of the group having received the Mix of probiotics are less marked by this infiltration.

During the development of obesity, the macrophages are recruited in the white adipose tissues while the regulating T-cells FoxP3⁺CD4⁺ leave these tissues.

FIG. 4 illustrates in overweight mice the action of the Mix on the inflammation of the adipose tissues.

As shown by FIG. 4A, in comparison with the control tissues LFD, the expression levels of several specific markers of monocytes/macrophages (F4/80; Cd68; Cd11b; and Cd11c) are increased in mice of the HFD group while the levels relating to the marker Foxp3 are reduced.

The administration of the mixture of probiotics Mix significantly reduces the expression induced by the HFD diet of the markers of monocytes/macrophages and increases the expression levels of the marker Foxp3.

The impact of the Mix mixture, and therefore of the probiotic strain according to the invention, on the recruitment of macrophages in adipose tissues is supported by the results illustrated in FIG. 4B.

According to the reduction in the recruitment of macrophages jointly with an increase of the accumulation of anti-inflammatory Treg lymphocytes, it is shown in this example that the epididymal adipose tissues in mice treated with the Mix mixture have an inflammation level less than the control tissues, as demonstrated by the reduced expression levels for the specific messenger RNAs of II6, Tnfα, II-1α, and II-17 (see FIG. 4C).

Further, it is shown that the treatment based on the Mix mixture limits the reduction in PPARy (both at the messenger RNA level and of the protein) induced by the HFD diet (see FIG. 4D).

### EXAMPLE 3

### Action of the Mix on the Metabolism of Lipids of the Small Intestine and the Production of Short Chain Fatty Acids (SCFA)

It is shown in this example that the expression levels of the genes (GPR41 and GPR43) which are responsible for the transport of short chain fatty acids (SCFAs for Short-Chain Fatty Acids) are decreased in the small intestine of mice subject to the HFD diet, which indicates that in the HFD group, the SCFAs are to a lesser extent capable of positively acting when the latter are less detected, while they are significantly increased in animals treated with the Mix mixture under the HFD diet (see FIG. 5B).

Vice versa, the administration of Mix has the effect of limiting the increase in the genes implied in the lipid metabolism induced by a fat food diet (see FIG. 5A).

### EXAMPLE 4

### Action of the Mix on the Microbiot and the Colonization level by Akkermansia muciniphila

An analyses of certain bacteria of the microbiot was conducted by quantitative PCR (qPCR) from the caecal contents of the mice which were subject to the HFD diet jointly or not with administration of the Mix, and on the other hand mice subject to the LFD diet jointly or not with administration of the Mix.

The results illustrated in FIG. 6 show a change in the composition of the microbiot of the HFD-Mix mice, with in particular a restoration at the colonization by Akkermansia muciniphila in mice subject to the HFD diet and treated with the probiotic strain or the mixture of probiotics according to the invention.

### EXAMPLE 5

### Action of the Mix on the Production of SFCA; Use of the SHIME Model

In this example, a dynamic model in vitro of simulation of the intestine, which is intended to reproduce the microbial ecosystem of the human intestine, was used.

As shown by the results from this simulation (see FIG. 5C), the use of the strain no. LMG P-28149 of the probiotic Bifidobacterium animalis ssp. Lactis allows an increase, within a period of 48h after inoculation, of the production of total SFCAs, promoting the production in the intestine of butyrate and propionate, two metabolites which promote satiation induced by the uptake of nutrients in human beings. The butyrate and propionate levels produced in the ascending colon associated with the SHIME reactor before (T0) and after (T24 h and T48 h) the incubation of the Mix mixture according to the invention are repeated in table 2. These data are expressed in mmol/L±SED.

TABLE 2

Period

| Products | T0 | T24 h | T48 h |
|---|---|---|---|
| Acetate | 3.53 ± 0.38 | 10.96 ± 0.91 | 3.10 ± 0.16 |
| Propionate | 1.10 ± 0.03 | 3.87 ± 0.51 | 7.07 ± 0.16 |
| Isobutyrate | 0.00 ± 0.00 | 0.24 ± 0.05 | 1.30 ± 0.05 |
| Butyrate | 0.69 ± 0.07 | 3.25 ± 0.27 | 12.27 ± 0.22 |
| Isovalerate | 0.11 ± 0.00 | 0.50 ± 0.05 | 2.44 ± 0.04 |
| Isocaproate | 0.00 ± 0.00 | 0.00 ± 0.00 | 0.00 ± 0.00 |
| Caproate | 0.00 ± 0.00 | 0.00 ± 0.00 | 0.00 ± 0.00 |
| Total | 5.44 ± 0.33 | 18.84 ± 1.46 | 26.19 ± 0.36 |

Demonstration of the Importance of Glutathion in the Culture Medium of the Strain Bifidobacterium animalis ssp. lactis No. LMG P-28149

FIGS. 8a and 8b illustrate the impact of the probiotic Bifidobacterium animalis ssp. lactis no. LMG P-28149 according to the invention on the weight gain over time depending on whether the probiotic was cultivated (FIG. 8a) or not cultivated (FIG. 8b) in a culture medium according to the invention.

As this may be seen by comparing FIGS. 8a and 8b and more particularly the HFD B. lactis curves, when a fat-rich food diet is associated with a taking of the composition according to the invention wherein the probiotic Bifidobacterium animalis ssp. lactis no. LMG P-28149 is obtained by cultivation in a culture medium comprising glutathion, a significant reduction in the weight gain overtime is observed (FIG. 8a), which is not the case during the administration of a composition according to the invention which comprises the probiotic obtained by cultivation in a culture medium not comprising glutathion (FIG. 8b).

It is quite understood that the present invention is by no means limited to the embodiments described above and that many modifications may be provided thereto without departing from the scope of the appended claims.

### LIST OF THE ACRONYMS

SOFA: Short chain fatty acid

EWAT: Epididymal white adipose tissues

GT(T): Glucose tolerance (Test)

HDL: High density lipids

H&E: Hematoxylin and eosin

HFD: Fat-rich diet

BMI: Body Mass Index

(HOMA)-IR: (homeostatic model) for appreciating insulin resistance

IT(T): Insulin tolerance (Test)

(V)LDL: (very) low density lipid

LFD: Low fat diet

NEFA: Non-esterified fatty acid

PBS: Saline phosphate buffer

PBMC: Mononucleated cells of peripheral blood

SCWAT: Subcutaneous white adipose tissues

SEM: Standard deviation relative to the mean

SHIME: Simulator of the Human Intestinal Microbial Ecosystem

WAT: White adipose tissues

1. A composition based on at least one probiotic for its use in the curative treatment of body weight gain in overweight human beings and/or in the preventive treatment of body weight gain in overweight human beings or having been overweight, characterized in that said probiotic is Bifidobacterium animalis ssp. lactis no. LMG P-28149.

2. The composition according to claim 1, comprising at least one additional probiotic selected from the group consisting of the following probiotics: Archaea, Firmicutes, Bacteroidetes, Proteobacteria, Actinobacteria, Verrucomicrobia, Fusobacteria, Metanobacteria, Spirochaetes, Fibrobacters, Deferribacteres, Deinococcus, Thermus, Cyanobacteria, Methanobrevibacterium, Lactobacillus, Peptostreptococcus, Ruminococcus, Coprococcus, Subdolingranulum, Dorea, Bulleidia, Anaerofustis, Gemella, Roseburia, Catenibacterium, Dialister, Anaerotruncus, Staphylococcus, Micrococcus, Propionibacterium, Enterobacteriaceae (non-pathogenic), Faecalibacterium, Bacteroides, Parabacteroides, Prevotella, Eubacterium, Akkermansia, Bacillus, Butyrivibrio, and Clostridium, or a combination thereof.

3. The composition according to claim 1, comprising a fungus and/or yeast strain selected from the group consisting of Saccharomyces, Candida, Pichia, Debaryomyces, Torulopsis, Aspergillus, Rhizopus, Mucor, and Penicillium.

4. The composition according to claim 1, wherein said Bifidobacterium animalis ssp. lactis no. LMG P-28149 and optionally said at least one additional probiotic are encapsulated in an encapsulation carrier.

5. The composition according to claim 4, wherein said encapsulation carrier comprises at least one substance selected from the group consisting of alginate, chitosan, pectin, pullulan, gelatin, carrageenan, agar gel, or a combination thereof.

6. The composition according to claim 1, comprising at least one source of food selected from the group consisting of a monosaccharide, polysaccharide, amino acid, peptide, protein, vitamin, yeast extract, halide salt of an alkaline or earth-alkaline metal, antioxidant, glycerol, zinc acetate, zinc chloride, zinc lactate, ascorbic acid, citric acid, plant oil, milk fat or a combination thereof.

7. The composition according to claim 1, further comprising at least one prebiotic, thus forming a symbiotic composition.

8. The composition according to claim 1, comprising a first enteric coating covering said Bifidobacterium animalis ssp. lactis no. LMG P-28149 and optionally said at least one additional probiotic.

9. The composition according to claim 8, wherein said first enteric coating is selected from the group consisting of ethylcellulose, hydroxypropylcellulose, carboxymethylcellulose, Eudragit®, or a combination thereof.

10. The composition according to claim 1, comprising a second external coating selected from the group consisting of alginate, chitosan, pectin, pullulan, gelatin, carrageenan, agar gel, cellulose, hemicellulose, ethyl cellulose, carboxymethyl cellulose, or a combination thereof.

11. The composition according to claim 1, further comprising one or several biocompatible excipients.

12. The non-therapeutic cosmetic use of a composition comprising at least a probiotic, in overweight human beings or having been overweight, said probiotic being Bifidobacterium animalis ssp. lactis no. LMG P-28149.

13. The non-therapeutic cosmetic use of a composition according to claim 12, said composition comprising at least one additional probiotic selected from the group consisting of the following probiotics: Archaea, Firmicutes, Bacteroidetes, Proteobacteria, Actinobacteria, Verrucomicrobia, Fusobacteria, Metanobacteria, Spirochaetes, Fibrobacters, Deferribacteres, Deinococcus, Thermus, Cyanobacteria, Methanobrevibacterium, Lactobacillus, Peptostreptococcus, Ruminococcus, Coprococcus, Subdolingranulum, Dorea, Bulleidia, Anaerofustis, Gemella, Roseburia, Catenibacterium, Dialister, Anaerotruncus, Staphylococcus, Micrococcus, Propionibacterium, Enterobacteriaceae (non-pathogenic), Faecalibacterium, Bacteroides, Parabacteroides, Prevotella, Eubacterium, Akkermansia, Bacillus, Butyrivibrio, and Clostridium, or a combination thereof.

14. The non-therapeutic cosmetic use of a composition according to claim 12, said composition comprising a fungus and/or yeast strain selected from the group consisting of Saccharomyces, Candida, Pichia, Debaryomyces, Torulopsis, Aspergillus, Rhizopus, Mucor, and Penicillium.

15. The non-therapeutic cosmetic use of a composition according to claim 12, wherein said Bifidobacterium animalis ssp. lactis no. LMG P-28149 and optionally said at least one additional probiotic are encapsulated in an encapsulation carrier.

16. The non-therapeutic cosmetic use of a composition according to claim 15, wherein said encapsulation carrier comprises at least one substance selected from the group consisting of alginate, chitosan, pectin, pullulan, gelatin, carrageenan, agar gel,. or a combination thereof.

17. The non-therapeutic cosmetic use of a composition according to claim 12, said composition comprising at least one food source selected from the group consisting of a monosaccharide, polysaccharide, amino acid, peptide, protein, vitamin, yeast extract, halide salt of an alkaline or earth-alkaline metal, antioxidant, glycerol, zinc acetate, zinc chloride, zinc lactate, ascorbic acid, citric acid, plant oil, milk fat, or a combination thereof.

18. The non-therapeutic cosmetic use of a composition according to claim 12, said composition further comprising at least one prebiotic, thus forming a symbiotic composition.

19. The non-therapeutic cosmetic use of a composition according to claim 12, said composition comprising a first enteric coating covering said Bifidobacterium animalis ssp. lactis no. LMG P-28149 and optionally said at least one additional probiotic.

20. The non-therapeutic cosmetic use of a composition according to claim 19, wherein said first enteric coating is selected from the group consisting of ethyl cellulose, hydroxypropyl cellulose, carboxymethylcellulose, Eudragit®, or a combination thereof.

21. The non-therapeutic cosmetic use of a composition according to claim 12, said composition comprising a second external coating selected from the group consisting of alginate, chitosan, pectin, pullulan, gelatin, carrageenan, agar gel, cellulose, hemicellulose, ethylcellulose, carboxymethylcellulose, or a combination thereof.

22. The non-therapeutic cosmetic use of a composition according to claim 12, said composition further comprising one or several biocompatible excipients.

23. A culture medium for the probiotic Bifidobacterium animalis ssp. lactis no. LMG P-28149 comprising at least one source of protein and at least one source of carbohydrates, said culture medium being characterized in that it further comprises glutathion.

24. The culture medium according to claim 23, characterized in that glutathion is present in a concentration comprised between 20 and 30 g/l of culture medium.

25. The culture medium according to claim 23, characterized in that said source of carbohydrates comprises at least one sugar or a mixture of sugars, which are selected from the group consisting of lactose, glucose, galactose, fructose, maltodextrin, starch, trehalose, maltotriose, and a combination thereof.

26. The culture medium according to claim 23, characterized in that it further comprises at least one aminoacid, for example, glucosamine or galactosamine.

27. The culture medium according to claim 23, characterized in that it further comprises at least one yeast extract.

28. A method for producing by fermentation the probiotic Bifidobacterium animalis ssp. lactis no. LMG P-28149, said method comprising at least one step for cultivating said probiotic in a culture medium according to claim 23.
